(19) United States
(12) Patent Application Publication
(10) Pub. No.: US 2025/0263434 A1
(43) Pub. Date: Aug. 21, 2025

(54) OLIGOPEPTIDE HAVING BLOOD PRESSURE REDUCING EFFECT, AND PREPARATION METHOD THEREFOR AND APPLICATION THEREOF

(71) Applicant: South China Agricultural University, Guangzhou (CN)

(72) Inventors: Yong CAO, Guangzhou (CN); Zeqi HE, Guangzhou (CN)

(73) Assignee: South China Agricultural University, Guangzhou (CN)

(21) Appl. No.: 17/778,034

(22) PCT Filed: Mar. 17, 2021
(86) PCT No.: PCT/CN2021/081215

§ 371 Date: May 19, 2022

(30) Foreign Application Priority Data
Mar. 31, 2020 (CN) 202010245463.X

(51) Int. Cl. C07K 7/06; A61K 38/00

(52) U.S. Cl. CPC C07K 7/06; A61K 38/00

(57) ABSTRACT

The present disclosure belongs to the technical fields of rice wine lees resource utilization and blood pressure reduction, and particularly relates to an oligopeptide having a blood pressure reducing effect and a preparation method therefor and an application thereof. In order to promote comprehensive utilization of rice wine lees, the oligopeptide having the blood pressure reducing effect is developed; the rice wine lees are used as raw materials, and separated and purified to obtain the oligopeptide having the blood pressure reducing effect. The oligopeptide has an amino acid sequence as shown in SEQ ID NO: 1, has a molecular weight of 720.857 Da, is high in purity and good in activity, can be artificially synthesized, safe and non-toxic, and is found by means of measurement of ACE inhibitory activity. The oligopeptide has a remarkable inhibition effect on angiotension converting enzyme, can be applied to the field of blood pressure reduction, and is beneficial to resource utilization of rice wine waste.

### REFERENCE TO SEQUENCE LISTING

The substitute sequence listing is submitted as a XML file filed via EFS-Web, with a file name of “Substitute_Sequence_Listing. TXT”, a creation date of Aug. 31, 2024, and a size of 554 bytes. The substitute sequence Listing filed via EFS-Web is a part of the specification and is incorporated in its entirety by reference herein.

### TECHNICAL FIELD

The present disclosure belongs to the technical field of resource utilization and blood pressure reduction of rice wine lees, and particularly relates to an oligopeptide having a blood pressure reducing effect, and a preparation method therefor and an application thereof.

### BACKGROUND ART

Hypertension is a main hazardous factor for cardiovascular diseases. Diets and life styles are important ways to prevent hypertension. Although there are differences between various blood pressure reducing mechanisms, a Rein-Angiotensin System (RAS) plays a crucial role in controlling blood pressure, and dysfunction of RAS can cause hypertension.

Artificially synthesized ACE inhibitors such as captopril and enalapril are the first kind of antihypertensive drugs. They can inhibit the conversion of inactive angiotensin I into active angiotensin II, thereby blocking the effect of RAS. However, they also inhibit the metabolic degradation of vasodilator kinins including bradykinin, thereby leading to specific side effects such as anaphylactic reaction, raised serum potassium level, dysgeusia and rash. Different from most of the vasodilators, Angiotensin-converting enzyme inhibitors (ACEI) can lower blood pressure without increasing heart rate. Captopril is the first non-peptide orally effective ACEI. Therefore, a suppressant ACE plays an important role in lowering blood pressure.

In China, deep development and comprehensive utilization of rice resources are still at an initial stage. In China, half ton of residues are not effectively utilized after each ton of rice is saccharified. the research level lags behind that of developed countries, so that their economic value has not been fully reflected. Therefore, the development of rice proteins and rice peptides has an extremely broad application prospect and space.

As a main byproduct of rice wine industry, the content of proteins in fresh rice wine lees is 28.1% (based on dry weight), so the fresh rice wine lees is a good protein resource. At present, 80 thousand-90 thousand tons of rice wine lees produced every year come from Guangdong Shunde distillery alone, and it is estimated that more than 400000 tons of rice wine lees are produced in the whole province every year.

However, in addition to a few of manufacturers using the dried rice wine lees to serve as feed, most of the manufacturers directly sell them to nearby farmers at a low price to serve as feed. Furthermore, the rice wine lees are directly discharged by some manufacturers, which not only wastes resources but also pollutes the environment. As the product obtained by fermenting rice, the rice wine lees contain a large number of available oligopeptides and proteins. Therefore, it is urgent to develop the field of comprehensive utilization of the rice wine lees, which can greatly improve the utilization value of the rice wine lees and reduce environmental pollution.

### SUMMARY

In order to overcome the defects in the prior art, the first objective of the present disclosure is to provide an oligopeptide having a blood pressure reducing effect.

The second objective of the present disclosure is to provide a preparation method of the above oligopeptide.

The third objective of the present disclosure is to provide an application of the above oligopeptide.

In order to realize the above objectives, the technical solution adopted by the present disclosure is as follows:

Provided is an oligopeptide, wherein the antihypertensive peptide has an amino acid sequence as shown in SEQ ID NO: 1.

In the present disclosure, the rice wine lees are filtered and centrifuged to remove impurities, and then subjected to separation and purification based on reversed-phase liquid chromatography for many times under the guidance of angiotensin converting enzyme inhibitory activity so as to obtain the oligopeptide. It is found by amino acid sequencing determination that the angiotensin has the amino acid sequence as shown in SEQ ID NO: 1 and a molecular weight of 720.857 Da. It is found by determination of angiotensin converting enzyme (ACE) inhibitory activity that the oligopeptide has a significant inhibition effect on ACE, and has a certain reference value in recycle of rice wine wastes and application of blood pressure reducing peptides.

Preferably, the oligopeptide has an inhibition effect on ACE. It is found by determination of ACE inhibitory activity that the oligopeptide obtained by separation and purification has an IC₅₀ of 280.00±5.77 μg/mL, and has a good inhibition effect on ACE. Meanwhile, it is shown that the oligopeptide has the blood pressure reducing effect.

The present disclosure also provides a preparation method of the above oligopeptide. That is to say, the rice wine lees are used as raw materials, and separated and purified to obtain the oligopeptide. Specifically, the preparation method comprises: the rice wine lees are filtered and centrifuged to remove impurities, and then subjected to separation and purification based on reversed-phase liquid chromatography for many times under the guidance of angiotensin converting enzyme inhibitory activity so as to obtain the oligopeptide. The separation and purification based on reversed-phase liquid chromatography comprises primary separation via a C18 preparative column, secondary separation via a C18 hydrophilic preparative column, and separation and purification via a reversed-phase high-performance liquid chromatography.

The present disclosure also provides application of the above oligopeptide in preparation of an angiotensin converting enzyme inhibitor.

The present disclosure also provides application of the above oligopeptide in preparation of an antihypertensive drug.

The present disclosure also provides an angiotensin converting enzyme inhibitor comprising the above oligopeptide. Of course, to improve the applicable range of a formulation, the formulation can also comprise other ingredients that can be applied to the field of blood pressure reduction. The formulation can be prepared into granules, capsules, tablets and other forms.

The present disclosure also provides an antihypertensive drug, comprising the above oligopeptide. Of course, to improve the applicable range of the drug, the drug can also comprise other ingredients that can be applied to the field of blood pressure reduction. The drug can be prepared into granules, capsules, tablets and other forms.

Compared with the prior art, the present disclosure has the beneficial effects:

In the present disclosure, the rice wine lees are used as raw materials, and separated and purified to obtain an oligopeptide having a blood pressure reducing effect. The oligopeptide has an amino acid sequence as shown in SEQ ID NO: 1, has a molecular weight of 720.857 Da, is high in purity and good in activity, can be artificially synthesized, and is safe and non-toxic. It is found by determination of ACE inhibitory activity that the oligopeptide has a significant inhibition effect on ACE, can be applied to the field of blood pressure reduction, and is beneficial to resource utilization of rice wine wastes.

### BRIEF DESCRIPTION OF THE DRAWINGS

FIG. 1 is a segmented sample loading diagram via a C18 preparative column after primary separation (D1-5).

FIG. 2 shows inhibitory activity of primarily separated sample (D1-5) via a C18 preparative column on ACE.

FIG. 3 is a segmented sample loading diagram via a C18 preparative column after secondary separation (S1-6).

FIG. 4 shows inhibitory activity of secondarily separated sample (S1-6) via a C18 preparative column on ACE.

FIG. 5 is a segmented sample loading diagram via RP-HCLP separation (H1-6).

FIG. 6 shows inhibitory activity of RP-HCLP separated sample (H1-6) on ACE.

FIG. 7 is secondary mass spectrum of component H6.

FIG. 8 is a PPSQ sequencing graph of component H6.

### DETAILED DESCRIPTION OF THE EMBODIMENTS

Next, specific embodiments of the present disclosure will be further described. It is noted that description of these embodiments is intended to help understanding the present disclosure but not limiting the present disclosure. In addition, technical features involved in various embodiments of the present disclosure described below can be mutually combined as long as they are not in conflict with each other.

Unless otherwise specified, test methods used in the following experimental examples are all conventional methods; the used materials, reagents and the like, unless otherwise stated, can be commercially available materials and reagents.

### Example 1 Separation of an Antihypertensive Peptide

### (1) Materials and Reagents

Materials: surplus base material after fermentation of rice wine in Shunde Hongli distillery (rice wine lees)

Reagents: see Table 1.

TABLE 1
Reagent table

| Reagents | Specification purity | Manufacturers |
|---|---|---|
| NaOH | Analytically pure | Qiqihar electrochemical plant of China |
| HCl | Analytically pure | Tianjin Fuyu Fine Chemical Co., Ltd., |
| | | China |
| NaCl | Analytically pure | Tianjin Qilun Chemical Technology Co., |
| | | Ltd, China |
| ACE | Analytically pure | American sigma company |
| HHL (Hippuryl-L-Histidyl-L-Leucine) | Analytically pure | American sigma company |
| TFA (trifluoroacetic acid) | Chromatographically | American sigma company |
| | pure | |
| C₂H₃N (CH₃CN) | Chromatographically | Tianjin Damao, China/Fisher |
| | pure | |
| Hippuric acid | Chromatographically | American sigma company |
| | pure | |
| Captopril | Analytically pure | American sigma company |

### (2) Determination Method of ACE Inhibitory Activity

### 2.1 Preparation of Reaction Solution

10 μL of ACE solution (0.25 U/mL, ACE enzyme was dissolved into pH 7 potassium phosphate buffer) was respectively added into a sample tube (B) and a blank tube (A), 10 μL of ACEI was added into the sample tube, 10 μL of buffer was added into the blank tube, samples were incubated for 5 min at 37° C., then 30 μL of HHL solution (6.5 mmol/mL, HHL was dissolved into pH 8.3 0.1 mol·L⁻¹ borate buffer, containing 0.3 mol·L⁻¹ NaCl) was added, the above materials reacted for 1 h at 37° C., and then 80 μL of 1.0 mol/L HCl was added to terminate the reaction, so as to obtain a reaction solution, seen in Table 2.

TABLE 2
Preparation of reaction solution

| | Control | Blank | Sample |
|---|---|---|---|
| Reagents | tube/μL | tube/μL | tube/μL |
| 1 mol/L HCl | 0 | 80 | 0 |
| (terminating agent) | | | |
| ACE (enzyme) | 10 | 10 | 10 |
| ACEI (inhibitor) | 0 | 0 | 10 |
| Buffer | 10 | 10 | 0 |
| Incubate for 5 min at 37° C. | | | |
| HHL (substrate) | 30 | 30 | 30 |
| React for 1 h at 37° C. | | | |
| 1 mol/L HCl (terminating | 80 | 0 | 80 |
| agent) | | | |

### 2.2 Chromatographic Conditions

Chromatographic column: ECOSIL C18 (260 mm×4.6 mm, 5 μm)

Mobile phase and chromatographic conditions: acetonitrile: ultrapure water=25:75 (including 0.1% (V/V) TFA), flow rate: 1 mL/min; detection wavelength: 228 nm; column temperature: 30° C.; injection volume: 20 μL.

### 2.3 Calculation of Results

Principle: HHL was rapidly decomposed under the catalysis of ACE to produce hippuric acid (Hip) and dipeptide (His-Leu, HL). Hippuric acid has the maximum absorption at 228 nm. When ACEI samples were added, the activity of the ACE enzyme was inhibited and the production amount of hippuric acid was reduced. Therefore, the inhibition rate of ACEI on ACE activity can be evaluated by measuring the production amount of hippuric acid by HPLC.

A calculation formula:

$${R}^{″} \frac{A - B - {A}_{0}}{A - {A}_{0}} \times 100 \%$$

In the formula, R: an inhibition rate (%) of ACEI sample on ACE;
A: a peak area of hippuric acid in control group
B: a peak area of hippuric acid in ACEI addition group
A0: a peak area of hippuric acid in blank group

Where, IC₅₀ is defined as a concentration of an inhibitor required when a half of ACE enzyme activity is inhibited under certain conditions. There was no a linear relationship between the inhibition rate and the concentration of the formulation, and therefore a curve of a relationship between the inhibition rate and the concentration of the formulation was firstly plotted, and then IC₅₀ was found out from the curve.

### (3) Pretreatment of Rice Wine Lees Raw Material

Rice wine lees was filtered with a 400-mesh filter cloth to remove filter residues, the filtrate was subjected to rotary evaporation and concentration at 55° C., then the concentrated filtrate was centrifuged for 15 min at 4000 rpm/min, supernate was retained, and then the supernate underwent freeze drying for 72 h at −80° C. to obtain an oligopeptide raw material.

### (4) Primary Separation Via C18 Preparative Column

The oligopeptide raw material was prepared into a 100 mg/mL solution, the solution was filtrated with a 0.45 μm filter membrane, and then separation and preparation was performed by using a Shimadzu PRC-ODS (K) chromatographic column (30 mm×250 mm, 15 μm, Shimadzu). The preparation conditions were as follows: mobile phase A was primary water (containing 0.1% TFA), mobile B was acetonitrile (containing 0.1% TFA), flow rate was 10 mL/min, injection volume was 5 mL, and monitoring was performed at 245 nm and 280 nm. An elution process is seen in Table 3.

TABLE 3
Elution process for primary separation

| | Concentration (%) of mobile |
|---|---|
| Retention time (min) | phase B |
| 0-30 | 10-34 |
| 30-45 | 34-90 |
| 45-60 | 90 |

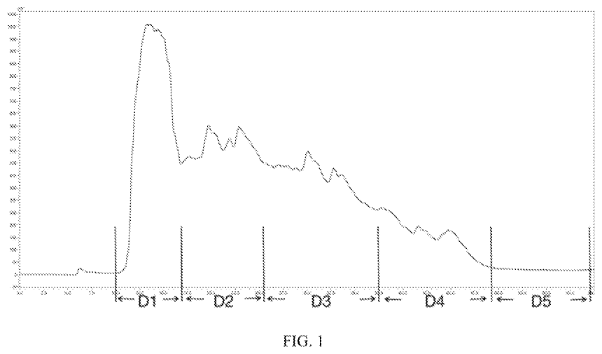

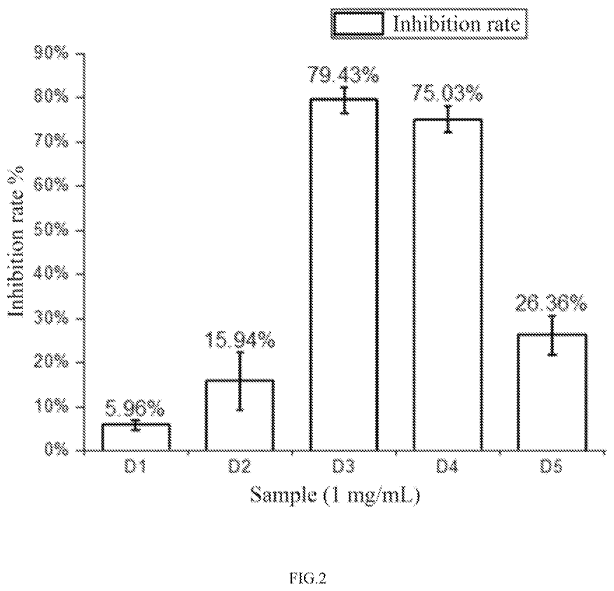

It can be seen from a separation and detection spectrum of FIG. 1 that the sample has about 8 peaks under the detection wavelength commonly used by the oligopeptide, that is, 214 nm. There are 5 sample loading segments including D1, D2, D3, D4 and D5 in total according to peak appearing time and peak shape similarity to respectively collect 5 components, then the 5 components were instantly concentrated and lyophilized in vacuum to reduce the degradation of the oligopeptide, and then the inhibitory activities of the 5 components on ACE enzyme were measured. It can be seen from inhibition rate measurement result from FIG. 2 that when the concentration of the sample is 1 mg/mL, the component D3 has a maximum inhibition rate being up to 79.43%, which is selected for separation and analysis in the next step.

### (5) Secondary Separation Via C18 Hydrophilic Preparative Column

The component D3 having the maximum inhibitory activity on ACE in the former step was prepared into a 30 mg/mL solution, the solution was filtered with a 0.45 μm filter membrane, and then the component D3 was re-separated and prepared by using a C18 hydrophilic preparative column. The chromatographic column is an ECOSIL C18 steel column (300 mm×20 mm, 10 μm, Germany). The preparation conditions were as follows: mobile phase A was primary water (containing 0.1% TFA), mobile B was acetonitrile (containing 0.1% TFA), flow rate was 10 mL/min, injection volume was 4 mL, and monitoring was performed at 245 nm and 280 nm. An elution process is seen in Table 4.

TABLE 4
Elution process for secondary separation

| | Concentration (%) of mobile |
|---|---|
| Time (min) | phase B |
| 0-5 | 15-19 |
| 5-45 | 19-23 |
| 45-47 | 23-90 |
| 47-57 | 90 |

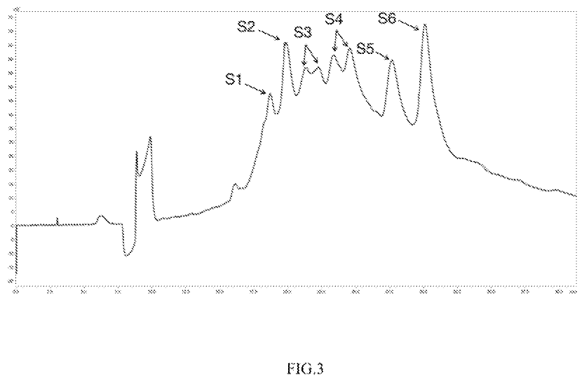

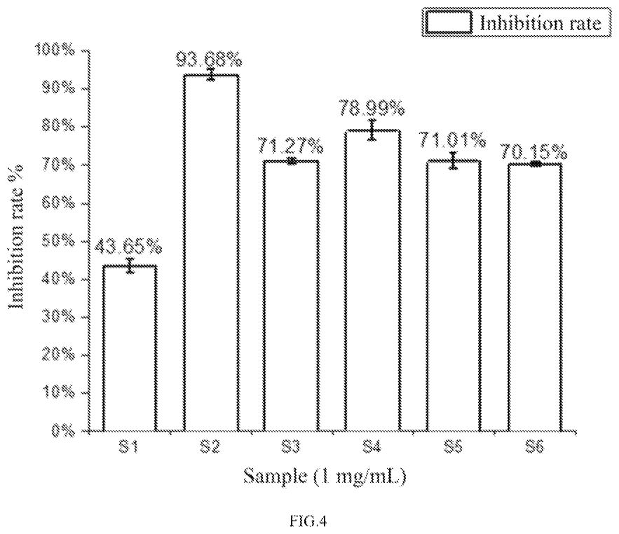

It can be seen from an separation and detection spectrum of FIG. 3 that there are 6 sample loading segments including S1, S2, S3, S4, S5 and S6 in total according to peak appearance situation and separation degree to respectively collect 6 components, then the inhibitory activity of each component on ACE enzyme was measured after the 6 components were concentrated and lyophilized. It can be seen from inhibition rate measurement result from FIG. 4 that through further separation, when the concentration of the sample is 1 mg/mL, the component S2 has a maximum inhibition rate being up to 93.68%. To explicit the high-activity peptide segment that plays an important role, the component S2 was separated and analyzed in the next step.

### (6) Separation and Purification Via Reversed-Phase High-Performance Liquid Chromatography (PR-HCLP)

The component S2 having the maximum inhibitory activity on ACE in the former step was prepared into a 20 mg/mL solution, the solution was filtered with a 0.45 μm filter membrane, and the component S2 was re-separated and prepared by using RP-HCLP. The chromatographic column is an ECOSIL C18 steel column (260 mm×4.6 mm, 5 μm, Germany). The analysis conditions were as follows: mobile phase A was primary water (containing 0.1% TFA), mobile B was acetonitrile (containing 0.1% TFA), flow rate was 1 mL/min, injection volume was 20 μL, and monitoring was performed at 245 nm and 280 nm. An elution process is seen in Table 5.

TABLE 5
Elution process via analysis chromatographic column

| | Concentration (%) of mobile |
|---|---|
| Retention time (min) | phase B |
| 0-10 | 5-15 |
| 10-45 | 15-20 |
| 45-60 | 90 |

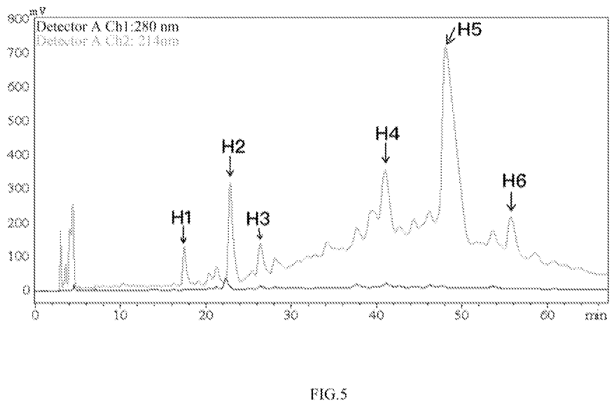

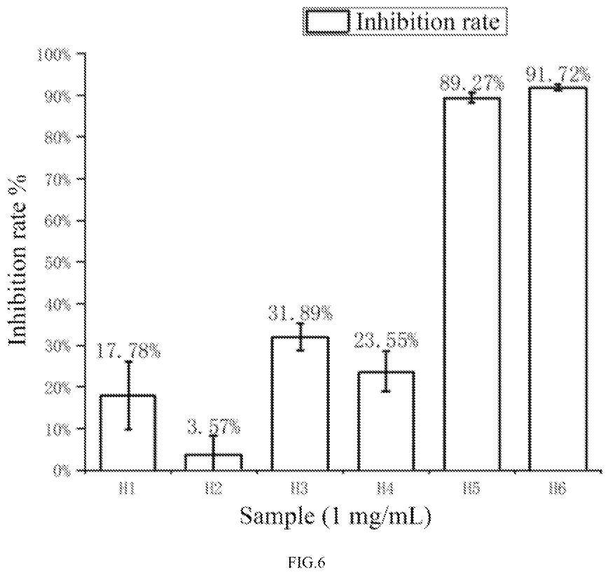

The purity of the sample after being separated and purified twice still needs to be further improved. After the separation process was adjusted, chromatographic peaks with good separation degrees and high response values were selected to be enriched. It can be seen from the HPLC spectrum shown in FIG. 5 that there are 6 peak loading samples including H1, H2, H3, H4, H5 and H6 in total according to peak appearing situations, the 6 samples were collected and subjected to rotary evaporation and concentration and freeze drying, and then the inhibition rate of each component on ACE was measured. It can be seen from inhibition rate measurement results in FIG. 6 that the inhibition rates of components H5 and H6 (1 mg/mL) on ACE are respectively 89.27% and 91.72% which are significantly higher than those of other 4 components (p<0.05). Therefore, the oligopeptides H5 and H6 are subjected to follow-up study in detail.

### (7) Determination of Antihypertensive Oligopeptide Structure

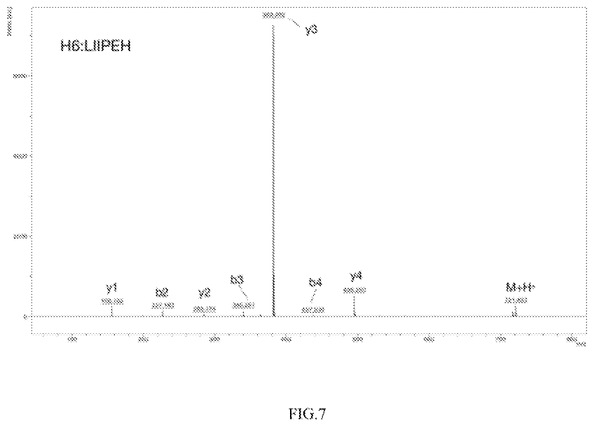

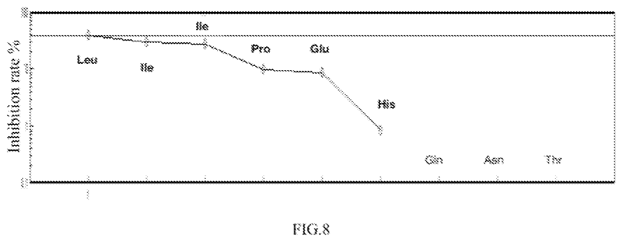

To study the compositions of the H6 antihypertensive peptide, the molecular weights of the above two purified peptides were identified by using a matrix assisted laser desorption ionization flight time mass spectrometer (MOLDI-TOF-MS/MS), the relative molecular weight of the oligopeptide H6 was 720.857 Da by subtracting the mass unit of attached protons from the charge mass ratio. However, since leucine has the same molecular weight as those of isoleucine, they were difficultly distinguished from the mass spectrum. Therefore, the amino acid composition of the peptide was determined by amino acid sequencing meter (PPSQ), and the N-terminal absolute sequence of the oligopeptide can be measured by PPSQ. According to a secondary mass spectrum (FIG. 7) and an amino acid sequencing analysis diagram (FIG. 8), the sequence of the oligopeptide H6 was finally determined as Leu-Ile-Ile-Pro-Glu-His (LIIPEH). The oligopeptide H6 was LIIPEH (antihypertensive oligopeptide H6) separated from food-derived natural products, and had high ACE inhibitory activity.

### Experimental Example 2 Artificial Synthesis of Antihypertensive Oligopeptides and Evaluation on Inhibitory Activity of ACE

To reversely verify the antihypertensive oligopeptide H6 and characterize its in-vitro inhibition effect on ACE in detail, a high-purity antihypertensive oligopeptide H6 (purity>98%) was synthesized by Shanghai jiepeptide Biotechnology Co., Ltd. and its activity was compared with the activity of the oligopeptide separated at each stage. The inhibitory activity evaluation test of ACE was carried out according to the determination method of the ACE inhibitory activity. The IC₅₀ values obtained by the test are seen in Table 6.

It can be seen from the results in Table 6 that the antihypertensive oligopeptide H6 separated by the present disclosure has a good ACE inhibitory activity, and it is reversely verified through artificial synthetic means that the two oligopeptides truly have the blood pressure reducing effect, so the two oligopeptides are expected to be applied to the field of blood pressure reduction and lay a foundation for the application of natural product antihypertensive peptides in the future. In addition, the IC₅₀ value of the oligopeptide is decreased after separation in each step, and the IC₅₀ value of the artificially synthesized oligopeptide is much lower than that of the separated oligopeptide, which is closely related to the purity of the oligopeptide.

TABLE 6
IC₅₀ values of synthesized oligopeptides and
oligopeptides in each separation stage

| Test samples | IC₅₀ (μg/mL) |
|---|---|
| Oligopeptide raw material after treatment | 806.67 ± 58.97 |
| Primarily separated oligopeptide D3 | 643.33 ± 17.64 |
| Secondarily separated oligopeptide S2 | 479.00 ± 59.36 |
| RP-HCLP separated monomer H6 | 280.00 ± 5.77 |
| Artificially synthesized monomer H6 | 56.67 ± 12.02 |

The embodiments of the present disclosure have been described in detail above, but the present disclosure is not limited to the described embodiments. Various changes, modifications, substitutions and modifications made by those skilled in the art without departing from the principle and spirit of the present disclosure are still fall within the protection scope of the present disclosure.

1. An oligopeptide having an amino acid sequence as shown in SEQ ID NO: 1.

2. The oligopeptide according to claim 1, wherein the oligopeptide has an inhibition effect on angiotensin converting enzyme.

3. The oligopeptide according to claim 1, wherein the oligopeptide has a blood pressure reducing effect.

4. A preparation method of the oligopeptide according to claim 1, wherein the rice wine lees are used as raw materials, and separated and purified to obtain the oligopeptide.

5. The preparation method according to claim 4, wherein the rice wine lees are filtered and centrifuged to remove impurities, and then subjected to separation and purification based on reversed-phase liquid chromatography for many times under the guidance of angiotensin converting enzyme inhibitory activity so as to obtain the oligopeptide.

6. The preparation method according to claim 5, wherein the separation and purification based on reversed-phase liquid chromatography comprises primary separation via a C18 preparative column, secondary separation via a C18 hydrophilic preparative column, and separation and purification via a reversed-phase high-performance liquid chromatography.

7. Application of the oligopeptide according to claim 1 in preparation of an angiotensin converting enzyme inhibitor.

8. Application of the oligopeptide according to claim 1 in preparation of an antihypertensive drug.

9. An angiotensin converting enzyme inhibitor comprising the oligopeptide according to claim 1.

10. An antihypertensive drug comprising the oligopeptide according to claim 1.

11. A preparation method of the oligopeptide according to claim 2, wherein the rice wine lees are used as raw materials, and separated and purified to obtain the oligopeptide.

12. A preparation method of the oligopeptide according to claim 3, wherein the rice wine lees are used as raw materials, and separated and purified to obtain the oligopeptide.

13. Application of the oligopeptide according to claim 2 in preparation of an angiotensin converting enzyme inhibitor.

14. Application of the oligopeptide according to claim 3 in preparation of an angiotensin converting enzyme inhibitor.

15. Application of the oligopeptide according to claim 2 in preparation of an antihypertensive drug.

16. Application of the oligopeptide according to claim 3 in preparation of an antihypertensive drug.

17. An angiotensin converting enzyme inhibitor comprising the oligopeptide according to claim 2.

18. An angiotensin converting enzyme inhibitor comprising the oligopeptide according to claim 3.

19. An antihypertensive drug comprising the oligopeptide according to claim 2.

20. An antihypertensive drug comprising the oligopeptide according to claim 3.


SEQUENCE LISTING (1 sequence)

SEQ ID NO: 1 - protein, 6 aa, Artificial Sequence
  antihypertensive peptide
LIIPEH